(19) United States
(12) Patent Application Publication
(10) Pub. No.: US 2016/0095891 A1
(43) Pub. Date: Apr. 7, 2016

(54) COMPOSITION FOR PREVENTING AND TREATING INFLAMMATORY DISEASE, CONTAINING OIL OF LITSEA JAPONICA AS ACTIVE INGREDIENT, AND METHOD FOR PREPARING SAME

(71) Applicants: HURUM CO.,LTD., Seoul (KR); JEJU TECHNOPARK, Jeju-do (KR)

(72) Inventors: Ju-Hyun CHO, Chungju (KR); In-Jae PARK, Cheonan (KR); Yong-Hwan JUNG, Seogwipo (KR); Goo-Hee CHOI, Cheongju (KR); Weon-Jong YOON, Jeju (KR); Sung-Hwan PARK, Jeju (KR); Seung-Hee SIN, Jeju (KR); Ji-Hyun YUN, Jeju (KR); Soon-Ok BAIK, Incheon (KR)

(21) Appl. No.: 14/889,611

(22) PCT Filed: Apr. 30, 2014
(86) PCT No.: PCT/KR2014/003883

§ 371 Date: Nov. 6, 2015

(30) Foreign Application Priority Data
May 6, 2013 (KR) 10-2013-0050388

(51) Int. Cl. A61K 36/54

(52) U.S. Cl. CPC A61K 36/54; A61K 2236/35

(57) ABSTRACT

The present invention relates to a composition for an inflammatory disease, using Litsea japonica, and more specifically to a composition for preventing and treating an inflammatory disease, containing the oil derived from the fruits or the seeds of Litsea japonica, and a method for preparing the same. The present invention uses the fruits (excluding the seeds) or the seeds of Litsea japonica and obtains the oil, which is used as an active ingredient of a composition for preventing and treating an inflammatory disease, by using n-hexane, wherein the oil is obtained by: separating the fruits (excluding the seeds) of Litsea japonica from the seeds; injecting n-hexane, which is a solvent, into the fruits (excluding the seeds) or the seeds, 9-11 times greater than the weight of each: stirring the same for 24 hours at room temperature and extracting the same; and removing a hexane layer.

### TECHNICAL FIELD

The present invention relates to a composition for an inflammatory disease using a Litsea japonica, and in particular to a composition for preventing and treating an inflammatory disease, which composition contains as an active ingredient an oil derived from the fruits and seeds of Litsea japonica and a method for preparing the above composition.

### BACKGROUND ART

The Litsea japonica used in the present invention is an evergreen is leaf arbor and is mainly growing on seashore and slope the altitudes of which are lower than 700 meters in a vertical direction.

According to the research conducted with respect to the chemical configuration of such a plant, it is reported that various kinds of essential oils, fatty acids, lactones, alkaloids and terpenoids are contained.

Hamabiwalactone A, hamabiwalatone B, akolactone B, litsealactone A and litsealactone B which were known as the kinds of lactones were separated.

In addition, as a result of the research on anti-complement activity of epicatechin, afzelin, quercitrin and tiliroside which are flavonoids ingredients separated from leaves, it is reported that the tiliroside has showed the strongest inhibition effect on a complement system.

According to the research wherein Litsea japonica leaf 80% EtOH extract was fragmented, and anti-oxidation (DPPH and superoxid radical inhibition activity was good at the DPPH, Xanthine Oxidase, Superoxide radical→fragmented material EtOAC layer) and LDH, NO, PGE2 (anti-inflammatory) were measured (YOON, Wonjong, et. al. 2010, J, Korean Soc. Appl. Biol. Chem. 53(1): p27-32) using the fragmented material, and after flavonoid species of leaf was separated (Epicatechin, Afzlin, Quercitrin, Tiliroside, Myricitrin, Kaempferol, Quercetin, Myricetin) using a silica column, the anti-complement system activity is was measured (Sun Young Lee, et. al., 2005, PHYTOTHERAPY RESEARCH, 19: p.273-276), and the lipid peroxide production inhibition was measured after Litsea japonica was subjected to extraction using methanol from edible and medicine plants (PARK, Jong-chul et. al., 1997, J. Korean Soc. Food Sci. Nutr. 26(6): p.1159-1163), it was reported that in case of Litsea japonica, the activity showed about 18% inhibition effect at a concentration of 10⁻¹ mg/mL.

In addition, according to the research (2009, KIM, Elbira, et. al., Ykhak Hoeji, 53(1): p.6-11) conducted on HL-60 leukemia cell apoptosis induction effect of Litsea japonica, which was partially carried on the Litsea japonica, it was extracted using Litsea japonica leaves and 80% of ethanol, and as a result of the HL-60 (cell toxicity)-MTT measurement, there was not any toxicity on normal cells, and HL-60 (leukemia cells) cell proliferation inhibition effects appeared, and there was a proliferation inhibition effect by inducing apoptosis by reducing the revelation of Bcl-2 and activating caspase through the DNA fragmentation analysis and the western blot analysis.

As a conventional art of the patent document, there are a technology on a composition for preventing and treating a cancer disease containing Litsea japonica as an active ingredient disclosed in (KR) Korean patent publication number 10-2009-0091477 (publication date: Aug. 28, 2009), and a technology on a composition for preventing and treating a diabetes mellitus is complication containing Litsea japonica extract or its fragmented material as an active ingredient disclosed in (KR) Korean patent registration number 10-1243243 (registration number: Mar. 7, 2012).

### DISCLOSURE OF INVENTION

### Technical Problem

Accordingly, it is an object of the present invention to provide a composition for preventing and treating an inflammatory disease containing oil of Litsea japonica as an active ingredient and a method for preparing the same which have effect on preventing and treating an inflammatory disease.

It is another object of the present invention to provide a composition for preventing and treating an inflammatory disease containing oil of Litsea japonica as an active ingredient and a method for preparing the same which have been invented by confirming that the oil from Litsea japonica (fruits or seeds) have a good effect on inhibiting the production of IL-1β, NO and TNF-α which are known as an inflammatory factor while screening from a natural material so as to develop a material having a good effect on preventing and treating an inflammatory disease.

### Technical Solution

To achieve the above objects, there is provided a composition for preventing and treating an inflammatory disease containing oil of Litsea japonica as an active ingredient and a method for preparing the same wherein the composition for preventing and treating an inflammatory disease can be derived using n-hexane from the fruits (excludeding seeds) or seeds of Litsea japonica.

The oil from the fruits (excludeding seeds) of Litsea japonica and the oil from the seeds of Litsea japonica each may be used in a singular form or may be used together in a combined form.

The oil from Litsea japonica may be extracted in various ways. In the present invention, it may be derived in such a way that the fruits (excludeding seeds) and the seeds of Litsea japonica are separated from each other, and the fruits (excludeding seeds) or the seeds are inputted 9˜11 times as compared with their weights in n-hexane which is a solvent, and the to materials are agitated for 24 hours at room temperature, thus extracting and finally obtaining the oil after a hexane layer is removed.

### Advantageous Effects

The oil from Litsea japonica contains Beta-cubebene (22.96%), Beta-selinene (11.23%), Gamma-Elemene (10.21%), Beta-Caryophyllene (7.37%), and as a result of experiment, it shows an IL-1β production inhibition ability, a NO production inhibition ability, a TNF-α production inhibition ability, whereupon the oil from Litsea japonica may be used as an active ingredient for a composition for preventing and treating an inflammatory disease.

### BRIEF DESCRIPTION OF DRAWINGS

FIG. 1 is a graph showing an IL-β production inhibition ability of the oils from fruits and seeds of Litsea japonica according to an exemplary embodiment of the present invention.

FIG. 2 is a graph showing a NO production inhibition ability of the oils from fruits and seeds of Litsea japonica according to an exemplary embodiment of the present invention.

FIG. 3 is a graph showing a TNF-α production inhibition ability of the oils from fruits and seeds of Litsea japonica according to an exemplary to embodiment of the present invention.

FIG. 4 is a chromatogram graph of the oil from fruits of Litsea japonica according to an exemplary embodiment of the present invention.

### BEST MODES FOR CARRYING OUT THE INVENTION

The present invention is directed to a composition for preventing and treating an inflammatory disease and a method for preparing the same which may be used to prepare and treat an inflammatory disease wherein the composition contains as an active ingredient the oil from Litsea japonica.

The oil from Litsea japonica which is an active ingredient of a composition according to the present invention may be obtained in such a way to separate into fruits (excludeding seeds) and seeds (seeds in the fruits), thus obtaining oil, and it may be prepared by the method below.

It is obvious that the oil may be obtained from the fruits (fruits and seeds remain unseparated) including seeds, more specifically, in a state where the fruits and seeds of Litsea japonica are not separated yet.

In the exemplary embodiment of the present invention, the fruits and seeds are separated in an effort to just obtain oil so as to seek any application of various compositions, for example, medicine and medical supplies, foods, etc., and alternatively the oil may be obtained from fruits containing seeds, which is also included in a scope of the present invention.

As a method for extracting oil from fruits (excludeding seeds) or seeds (seeds in the fruits) or fruits containing seeds, there are a supercritical extraction method, a distillation method, a compression method, etc. in addition to the extraction method which uses n-hexane corresponding to an exemplary is embodiment of the present invention. The oil can be obtained using such methods. If the distillation method or the compression method is used, it has a disadvantage in the way that the amount of extracted oil may be smaller than the extraction method which uses n-hexane corresponding to the exemplary embodiment of the present invention.

The n-hexane, which is a solvent, is inputted 9˜11 times (preferably 10 times) as compared to weight, into the oil from the fruits (excludeding seeds) of Litsea japonica and the dried fruits (excludeding seeds) of Litsea japonica, and the material is agitated for 24 hours at room temperature (15˜25° C.) and is subjected to extraction, and a hexane layer is removed, thus finally preparing the oil from the fruits of Litsea japonica.

The solvent n-hexane is inputted 9˜11 times (preferably 10 times) as compared to weight, into the oil from the seeds of Litsea japonica and the dried seeds of Litsea japonica, and the material is agitated for 24 hours at room to temperature (15˜25° C.) and is subjected to extraction, and a hexane layer is removed, thus finally preparing (obtaining) the oil from the seeds of Litsea japonica.

The fruits oil and seeds oil of Litsea japonica each may be used in a singular form or may be used after they are mixed at a predetermined ratio.

The oils from the fruits (excludeding seeds) and seeds of Litsea japonica according to the present invention may have even with a low concentration a very good effect on inhibiting the productions of TNF-α, IL-1β NO which are known as an inflammatory factor. To this end, the oils from fruits (excludeding seeds) and seeds of Litsea japonica according to the present invention may be used to prepare a composition (raw material) for medicine and medical supplies which are useful to prevent or treat an inflammatory disease and a health-related functional food.

The composition of the present invention may contain, together with the oils from the fruits (excludeding seeds) and seeds of Litsea japonica, one or more known active ingredients which have effect on preventing or treating an inflammatory disease.

For the sake of administration, the composition of the present invention may be prepared by using one or more carrier which is pharmaceutically acceptable, which is provided in addition to the above active ingredient.

The pharmaceutically acceptable carrier may be formed of any of saline solution, disinfected water, Ringers solution, buffered saline solution, dextrose solution, maltodextrin solution, glycerol, ethanol and a combination of one or more of the above components.

In order to prevent or treat an inflammatory disease, the composition of the present invention may be used in a singular form or in a combination of methods which use surgery, hormone treatment, medicine-based treatment and a biological reaction regulator.

The composition of the present invention may be used in such a way that it is added to a health function food so as to improve an inflammatory disease.

If the oil from fruits (excludeding seeds) or seeds of Litsea japonica of the present invention is used as a food additive, the oil from fruits (excludeding seeds) or seeds of Litsea japonica may be directly added and used or the fragmented material thereof may be used or it may be used together with other foods or food components. It may be appropriately used based on a typical method.

As examples of such foods which may be added, there are meats, sausage, breads, candies, snacks, confectionary, pizza, ramen, gums, dairy products including ice creams, various soups, drinks, teas, drink products, alcohol drinks, combined vitamins, etc. which are not limited thereto, and all kinds of health function foods based on typical meanings are all included.

### MODES FOR CARRYING OUT THE INVENTION

The exemplary embodiments of the present invention will be described for the sake of better understandings of the present invention. The exemplary embodiments below are provided for the sake of better understandings of the present invention, not intended to limit the contents of the present invention by the disclosed exemplary embodiments.

### Embodiment 1

### Preparation of Oil from Fruits (Excludeding Seeds) of Litsea Japonica

5000 ml of n-hexane was added to 500 g of the fruits (excludeding seeds) of the frozen and dried Litsea japonica, and the mixture was agitated for 24 hours at room temperature (20° C.) and was subjected to extraction, and then hexane was removed, thus obtaining 95 g of oil from the fruits (excludeding seeds) of Litsea japonica.

### Embodiment 2

### Preparation of Oil from Seeds of Litsea Japonica

5000 ml of n-hexane was added to 500 g of the seeds of the frozen and dried Litsea japonica, and the mixture was agitated for 24 hours at room temperature (20° C.) and was subjected to extraction, and then hexane was removed, thus obtaining 46 g of oil from the seeds of Litsea japonica.

### Experiment Example 1

Component analysis on oil from fruits (excludeding seeds) of Litsea japonica.

The experiment for component analysis on the oil from fruits (excludeding seeds) of Litsea japonica obtained according to the exemplary embodiment 1 was carried out.

Experiment method: The device was Shimadzu, QP2010Plus, and the column was RTX-5(30 m×0.25 mm, 0.25 μm), and as for other conditions during the analysis, the injection temperature was 250° C., the detector temperature was 250° C., the mass range (m/z) was 30˜400, and the injection volume was 1 μL.

A result of the experiment: a result seen in FIG. 4 and Table 1 was obtained by analyzing the oil from fruits (excludeding seeds) of Litsea japonica obtained according to the first exemplary embodiment, and it was confirmed that the oil contained Beta-cubebene (22.96%), Beta-selinene (11.23%), Gamma-Elemene (10.21%) and Beta-caryophyllene (7.37%).

The table 1 below is an analysis table of oil from fruits of Litsea japonica obtained according to the first exemplary embodiment

TABLE 1

| Compound name | Area (%) | peak | Remarks |
|---|---|---|---|
| Beta-cubebene | 22.96 | 14 | |
| Beta-selinene | 11.23 | 15 | |
| Gamma-Elemene | 10.21 | 16 | |
| Beta-Caryophyllene | 7.37 | 8 | |

### Experiment Example 2

Measurement (1) of effect on anti-inflammation of oils from fruits or seeds of Litsea japonica according to the present invention: IL-1β production inhibition ability.

In order to confirm any effect on anti-inflammation of oils from fruits (excludeding seeds) and seeds of Litsea japonica obtained according to the first and second exemplary embodiments, an experiment was carried out to measure any effect on inhibiting IL-1β production.

Experiment method: In order to evaluate an IL-1β production inhibition ability, the samples of oils from fruits and seeds of Litsea japonica was preprocessed for 1 hour before Raw 264.7 cell (1×10⁶ cell/mL) which was a to mouse macrophage was stimulated, and it was simulated with LPS (1 μg/mL), and after 24 hours, its amount was measured using the ELISA method on a cell culture supernatant, and for the ELISA, the mouse ELISA set (IL-1β) was purchased from the eBioscience (USA), and the experiment was carried out.

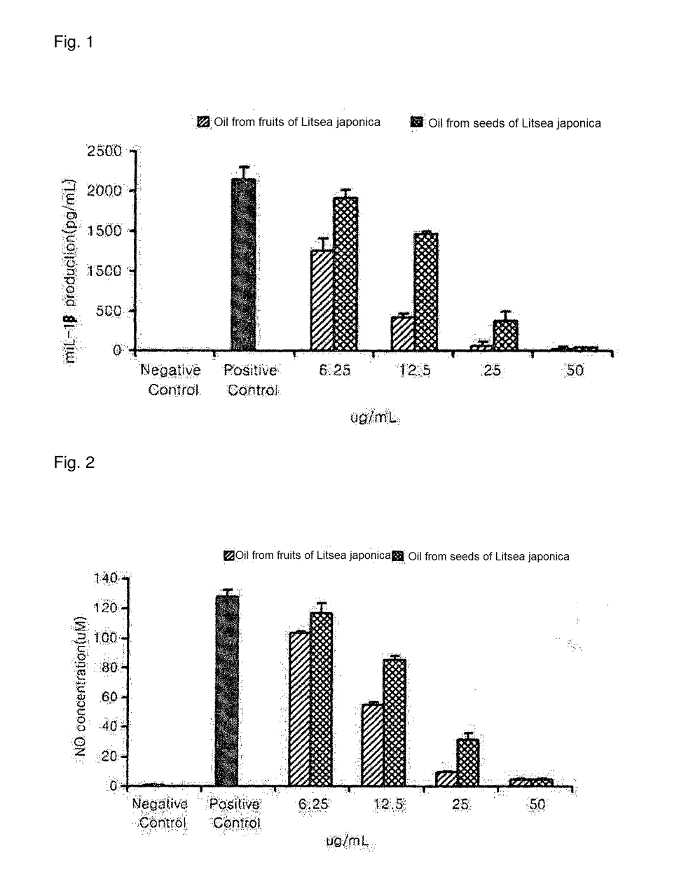

A result of the experiment: A result of the measurement on the IL-6 is production ability with respect to the oils from fruits and seeds of Litsea japonica obtained according to the first and second exemplary embodiments of the present invention is shown in FIG. 1 and Table 2. As a result, it was confirmed that the IL-1β production inhibition of the oil sample from fruits of Litsea japonica was better than the oil sample from the seeds, and the IL-6 production ability was inhibited dependent on the concentration. The refined oil sample from fruits showed 96% inhibition ability at a concentration of 25 ug/mL, and the refined oil sample from seeds showed a 82% inhibition ability, which both represent a good inhibition effect.

The Table 2 below shows an anti-inflammation effect based on the IL-1β production capability measurement on the oils from fruits and seeds of Litsea japonica obtained according to the first and second exemplary embodiments.

TABLE 2

| Classification | | IL-1β production (pg/ml) | Remarks |
|---|---|---|---|
| Control | Negative | 14.4 ± 1.5 | |
| | Positive | 2157.3 ± 163.5 | |
| First | 6.25 μg/mL | 1266.5 ± 156.7 | |
| embodiment | 12.50 μg/mL | 440.4 ± 21.8 | |
| | 25.00 μg/mL | 81.5 ± 25.3 | |
| | 50.00 μg/mL | 37.3 ± 8.7 | |
| Second | 6.25 μg/mL | 1922.8 ± 102.8 | |
| embodiment | 12.50 μg/mL | 1469.8 ± 30.2 | |
| | 25.00 μg/mL | 379.5 ± 106.0 | |
| | 50.00 μg/mL | 42.1 ± 1.5 | |

### Experiment Example 3

The measurement (2) of anti-inflammation effect on the oils from fruits or seeds of Litsea japonica according to the present invention: Evaluation on NO production inhibition ability

In order to confirm the anti-inflammation effect of the oils from fruits (excludeding seeds) and seeds of Litsea japonica obtained according to the first and second exemplary embodiments, an experiment for evaluating the NO production inhibition ability was carried out.

Experiment method: In order to measure a NO production inhibition ability, the oils from fruits and seeds of Litsea japonica was preprocessed for 1 hour before Raw 264.7 cell (1×10⁶ cell/mL) which was a mouse macrophage was stimulated, and it was simulated with LPS (1 μg/mL), and after 24 hours, the absorbance was measured at a concentration of Nitric oxide on a cell culture supernatant and at 540 nm using the NO detection kit (Intron biotechnology, Korea).

A result of the experiment: A result of the evaluation on the NO production ability with respect to the oils from fruits and seeds of Litsea japonica obtained according to the first and second exemplary embodiments of the present invention is shown in FIG. 2 and Table 3. As a result, it was confirmed that the NO production inhibitions of the oils from both the fruits and seeds of Litsea japonica were obtained dependent on the concentration.

The refined oil sample from fruits of Litsea japonica showed a 91% inhibition ability at a concentration of 25 ug/mL, and the refined oil sample from seeds of Litsea japonica showed a 74% inhibition ability, which both represent a good inhibition effect.

The table 3 below is a NO production inhibition ability measurement table of oils from fruits and seeds of Litsea japonica obtained according to the first and second exemplary embodiments.

TABLE 3

| Classification | | NO concentration (μM) | Remarks |
|---|---|---|---|
| Control | Negative | 1.0 ± 0.3 | |
| | Positive | 127.5 ± 4.6 | |
| First | 6.25 μg/mL | 103.5 ± 1.3 | |
| embodiment | 12.50 μg/mL | 55.3 ± 1.5 | |
| | 25.00 μg/mL | 10.4 ± 0.3 | |
| | 50.00 μg/mL | 5.2 ± 0.3 | |
| Second | 6.25 μg/mL | 116.7 ± 6.9 | |
| embodiment | 12.50 μg/mL | 84.8 ± 3.4 | |
| | 25.00 μg/mL | 32.0 ± 3.7 | |
| | 50.00 μg/mL | 4.8 ± 0.3 | |

### Experiment Example 4

The measurement (3) of anti-inflammation effect on the oils from fruits or seeds of Litsea japonica according to the present invention: Evaluation on TNF-α production inhibition ability

In order to confirm the anti-inflammation effect of the oils from fruits (excludeding seeds) and seeds of Litsea japonica obtained according to the first and second exemplary embodiments, an experiment for evaluating the TNF-α production inhibition ability was carried out.

Experiment method: In order to evaluate a TNF-α secretion inhibition ability, the oil samples from fruits and seeds of Litsea japonica were preprocessed for 1 hour before Raw 264.7 cell (1×10⁶ cell/mL) which was a mouse macrophage was stimulated, and it was simulated with LPS (1 μg/mL), and after 24 hours, its amount was measured using the ELISA method on a cell culture supernatant, and for the ELISA, the mouse ELISA set (TNF-α) was purchased from the eBioscience (USA), and the experiment was carried out.

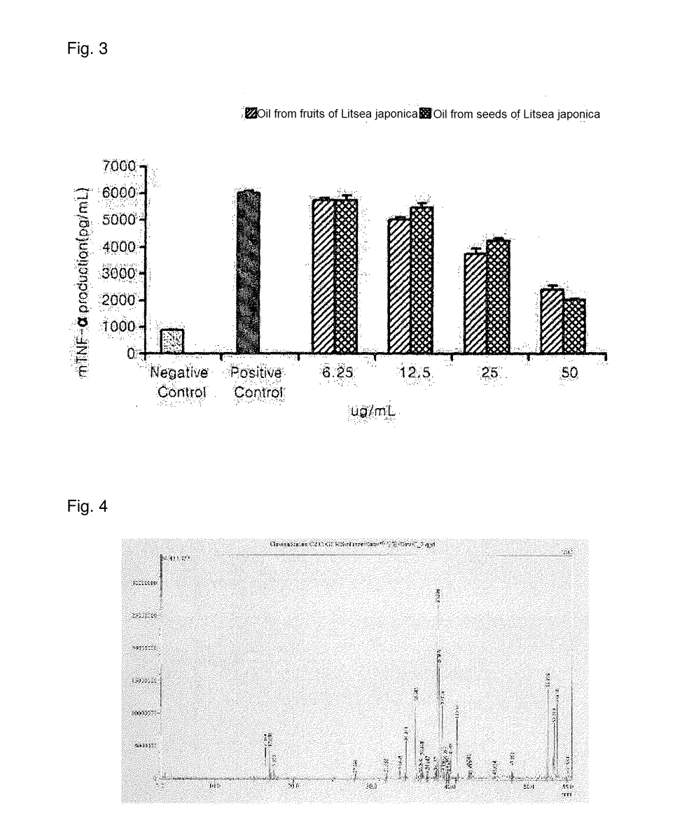

A result of the experiment: A result of the evaluation on the TNF-α production ability with respect to the oils from fruits and seeds of Litsea japonica obtained according to the first and second exemplary embodiments of the present invention is shown in FIG. 3 and Table 4. As a result, it was confirmed that the TNF-α production inhibitions of the oil samples from both the fruits and seeds of Litsea japonica were obtained dependent on the concentration. The oil sample from fruits of Litsea japonica showed a 37% inhibition ability at a concentration of 25 ug/mL, and the oil sample from seeds of Litsea japonica showed a 29% inhibition ability, which both represent a good inhibition effect.

The table 4 below is a TNF-α production inhibition ability measurement table of oils from fruits and seeds of Litsea japonica obtained according to the first and second exemplary embodiments.

TABLE 4

| Classification | | mTNF-α production (pg/ml) | Remarks |
|---|---|---|---|
| Control | Negative | 903 ± 3 | |
| | Positive | 6009 ± 74 | |
| First | 6.25 μg/mL | 5764 ± 66 | |
| embodiment | 12.50 μg/mL | 5044 ± 137 | |
| | 25.00 μg/mL | 3774 ± 180 | |
| | 50.00 μg/mL | 2416 ± 159 | |
| Second | 6.25 μg/mL | 5773 ± 148 | |
| embodiment | 12.50 μg/mL | 5479 ± 134 | |
| | 25.00 μg/mL | 4256 ± 93 | |
| | 50.00 μg/mL | 2056 ± 47 | |

### INDUSTRIAL APPLICABILITY

The oils extracted from the fruits (excludeding seeds) and seeds of Litsea japonica according to the present invention has a good effect on preventing and treating an inflammatory disease.

To this end, the oils from fruits and seeds of Litsea japonica according to the present invention may be very usefully used to prevent and treat an inflammatory disease.

1. A composition for preventing and treating an inflammatory disease, comprising: an oil extracted from Litsea japonica as an active ingredient.

2. The composition of claim 1, wherein the oil from Litsea japonica can be obtained from any of fruits excluding seeds, seeds and fruits containing seeds of Litsea japonica.

3. The composition of claim 2, wherein the oil from Litsea japonica is either a fruit oil from fruits excluding seeds of Litsea japonica or a seed oil from seeds of Litsea japonica, and the fruit oil and the seed oil can be used in a singular form or can be used mixed together.

4. The composition of claim 1, wherein the oil from Litsea japonica can be obtained by an extraction method which uses n-hexane as a solvent, a supercritical extraction method, a distillation method, or a compression method.

5. A method for preparing oil from Litsea japonica for preventing and treating an inflammatory disease, comprising:
inputting the fruits of Litsea japonica 9-11 times weight into n-hexane which is a solvent;
agitating the material for 24 hours at room temperature, and the material is subjected to extraction; and obtaining oil after removing a hexane layer.

6. The method of claim 5, wherein the fruits of Litsea japonica are separated into fruits excluding seeds and seeds, and oil can be prepared with the separated fruits excluding seeds and seeds.

7. The composition of claim 2, wherein the oil from Litsea japonica can be obtained by an extraction method which uses n-hexane as a solvent, a supercritical extraction method, a distillation method, or a compression method.

8. The composition of claim 3, wherein the oil from Litsea japonica can be obtained by an extraction method which uses n-hexane as a solvent, a supercritical extraction method, a distillation method, or a compression method.
